(19) United States
(12) Patent Application Publication
(10) Pub. No.: US 2026/0000351 A1
(43) Pub. Date: Jan. 1, 2026

(54) NON-INVASIVE METHODS OF MEASURING THE DEGREE OF CELLULAR ENERGY METABOLISM

(71) Applicant: AMAZON TECHNOLOGIES, INC., RENO, NV (US)

(72) Inventors: David HECKERMAN, Bellevue, WA (US); Frank Wilhelm SCHMITZ, Seattle, WA (US); Andreas CADUFF, Los Altos, CA (US); Michael MIYAMOTO, Mission Viejo, CA (US); Layne Christopher PRICE, Seattle, WA (US); Adrian NAPOLES, Bellevue, WA (US); Antje HEIT, Seattle, WA (US)

(21) Appl. No.: 18/725,262

(22) PCT Filed: May 24, 2024
(86) PCT No.: PCT/US2024/031033

§ 371 Date: Jun. 28, 2024

Related U.S. Application Data
(60) Provisional application No. 63/504,263, filed on May 25, 2023.

(51) Int. Cl. A61B 5/00; A61B 5/05; G01N 33/50

(52) U.S. Cl. CPC A61B 5/4866; A61B 5/05; G01N 33/5005

(57) ABSTRACT

This disclosure relates to methods of non-invasively measuring the degree of cellular energy metabolism. The methods disclosed herein comprise applying monitoring devices to a bodily surface or solution containing cells (e.g., a dermal surface of a subject), generating a radio frequency, detecting operating values of the circuitry of the device, maintaining contact between the monitoring device and the bodily surface or solution containing cells for a desired length of time, and collecting data that corresponds to the degree of cellular energy metabolism. Methods of this disclosure can be used to correlate the degree of cellular energy metabolism to other physiological or cellular phenomena (e.g., cellular respiration, cell viability).

### RELATED APPLICATIONS

The present application claims the benefit of U.S. Provisional Patent Application No. 63/504,263, filed May 25, 2023, the entire contents of which is incorporated herein by reference in its entirety.

### BACKGROUND

The measurement of cellular energy metabolism and cellular adenosine triphosphate (ATP) concentration are important for the diagnosis, treatment, and monitoring of various metabolic disorders. The ability to evaluate energy metabolism (e.g., cellular glucose metabolism, cellular energy metabolism (CEM), cellular ATP concentration) is essential for the treatment of diabetes (e.g., type 1 and type 2 diabetes) and allows for effective treatment with medication (e.g., insulin) or monitoring of dietary choices to avoid diabetic ketoacidosis, hyperosmolar hyperglycemic state (HHS), or loss of consciousness. Although blood glucose can be measured directly (e.g., blood collection, finger lancing), this is not ideal from a cost, safety, patient compliance, and data collection perspective. Collection of blood is often painful and requires repeated measurements every day. This process requires fresh sterile equipment (alcohol wipes, bandages, needles, lancets) for each glucose measurement. Additionally, measurements are made on a sample-by-sample basis and not available as a continuous series to determine blood glucose concentration baselines and trends more accurately. In view of these challenges, there is a need for a convenient, continuous, cost-effective, and painless method for determining cellular glucose processing, one type of cellular energy metabolism.

Measurement of interstitial glucose is one method to provide continuous measurements that can be correlated to blood glucose (see Cengiz, E. and Tamborlane W.V. A Tale of Two Compartments: Interstitial Versus Blood Glucose Monitoring (2009) Diabetes Technol Ther. 11 (Suppl 1), S11-16). As an example, the LIBRE (An interstitial glucose sensor, ABBOTT LABORATORIES) is a device that can detect interstitial glucose and infer blood glucose concentrations. While these and similar methods offer the ability to continuously track interstitial glucose, they require application of a device that punctures the skin. This may cause pain or irritation for the user and poses a risk for infection. Additionally, many of these devices must be replaced due to an exhausted battery or to create a new puncture site for reading due to sensor fouling, thus adding to patient cost.

Downstream of blood glucose concentrations, cellular energy metabolism is the utilization of energy sources (e.g., glucose, ketone bodies, pyruvate) to form adenosine triphosphate (ATP), the common energy unit for all cells. Overactive consumption of energy sources such as glucose can have detrimental effect on cellular vitality (e.g., oxidative stress, cell division, cell senescence) and serve as an indicator of physiological health conditions (e.g., obesity, metabolic fitness, aging, diabetes, cardiovascular damage, and cancer). Existing technology to directly measure the degree of cellular energy metabolism is limited to use in homogenous solutions of cells (e.g., suspended cells in media, adhered cells in media in a laboratory), and thus not suited for heterogenous measurements of cells on a solid surface (e.g., a bodily surface, the skin and underlying tissue). One approach to measuring degree of cellular energy metabolism in solution has been to use labeled reagents (e.g., radio-labeled reagents, fluorescent reagents, luminescence reagents) (see Csepregi R. et al. A One-Step Extraction and Luminescence Assay for Quantifying Glucose and ATP Levels in Cultured HepG2 Cells (2018) Int. J. Mol. Sci. 19, 9, 2670). However, these approaches require homogenous solutions of cells amenable to addition of these expensive reagents to cells. Moreover, alternatives to radio assay, luminescence assay, or fluorescence assay technology require strict control of the components in the media (e.g., carbon source, salt content), and require the measurement of multiple parameters including acidification of the media, carbon dioxide (CO₂) production, and/or oxygen consumption; the measurement of which require expensive and delicate instrumentation not suitable for use outside of a laboratory setting (see Zhang J. and Zhang Q., Using Seahorse Machine to Measure OCR and ECAR in Cancer Cells (2019), Cancer Metabolism: Methods and Protocols, Methods in Molecular Biology, 1928, 353-363).

Due to the disadvantages posed by existing detection methods, there is a need to provide a painless, cost-effective, and convenient method for non-invasively detecting cellular energy metabolism for the prevention of detrimental health outcomes.

This disclosure relates to methods of non-invasively measuring the degree of cellular energy metabolism by contacting (e.g., adhering, creating an interface with, touching, pressing, dipping, submerging) a monitoring device for detecting cellular energy metabolism (e.g., a cellular energy metabolism sensor device, a sensor device, a cellular energy metabolism monitoring device, a monitoring device) with a bodily surface or a solution comprising cells. The monitoring device for detecting the degree of cellular energy metabolism for methods described herein contains a sensor substrate that comprises i) one or more transmitting planes for transmitting a signal (e.g., a transmitted signal) at a radio frequency, and ii) a ground plane with a gap between the ground plane and the one or more transmitting planes for controlling the penetration depth of an electric field of the transmitted signal and adjusting sensitivity to adenosine triphosphate changes. Methods of non-invasively measuring the degree of cellular energy metabolism include the steps of a) contacting a monitoring device for detecting the degree of cellular energy metabolism to a bodily surface or a solution comprising cells, b) generating a transmitted signal at a radio frequency, c) measuring one or more operating values (e.g., electric power, resistance, capacitance, impedance, voltage, or current) of the circuitry of the monitoring device at the radio frequency of the transmitted signal, d) maintaining contact with a bodily surface or solution containing cells for a desired time, and e) collecting impedance data, voltage data, or current data from the sensor substrate of the monitoring device that indicates a degree of cellular energy metabolism (e.g., the change in cellular energy metabolism) for the bodily surface or the solution comprising cells.

The transmitted signal from the monitoring device can be of any frequency. In some embodiments, the radio frequency of the transmitted signal ranges from about 0.1 to about 250 MHz (e.g., about 0.5 to about 250 MHz). In some embodiments, the radio frequency of the transmitted signal ranges from about 40 MHz to about 75 MHz. In some embodiments, the radio frequency of the transmitted signal is about 64 MHz.

The monitoring device (e.g., the sensor substrate of the monitoring device) can contain transmitting planes of any number, shape, or dimensionality. The monitoring device (e.g., the sensor substrate of the monitoring device) can contain any number of transmitting planes. In some embodiments, the monitoring device comprises one transmitting plane. In some embodiments, the monitoring device comprises two transmitting planes. In some embodiments, the transmitting plane can be annular. In some embodiments, the transmitting plane can be bar shaped. In such embodiments, the transmitting plane can have a width ranging from about 0.25 mm to about 15 mm (e.g., about 1 mm to about 8 mm).

The monitoring device can measure the cellular energy metabolism for any desired length of time. In some embodiments, the desired length of time ranges from about 1 minute to about 24 hours. In some embodiments, the desired length of time ranges from about 1 minute to about 2 hours.

The monitoring device can be applied to a bodily surface by any type of attachment. In some embodiments, the monitoring device can be applied to a bodily surface by an attachment selected from the group consisting of a tape, a band, a wrap, an adhesive, and a combination thereof.

The sensor substrate used in methods of measuring the degree of cellular energy metabolism can be separated from the bodily surface or a solution comprising cells by an insulative layer. The insulative layer can be of any thickness (e.g., the thickness can range from about 0.1 μm to about 100 μm). In some embodiments, the thickness of the insulative layer is about 10 μm or less.

Methods of measuring the degree of cellular energy metabolism can predict, diagnose, or monitor a disease or disorder in a subject in need thereof. In such embodiments, the disease or disorder can be any disease or any disorder. In some embodiments, the method can predict, diagnose, or monitor a cardiovascular disease in a subject in need thereof. In such embodiments, the cardiovascular disease can be coronary artery disease, peripheral arterial disease, cerebrovascular disease, renal artery stenosis, or aortic aneurysm. In some embodiments, the method can predict, diagnose, or monitor a metabolic disease or disorder in a subject in need thereof. In such embodiments, the metabolic disease or disorder can be prediabetes, type 1 diabetes, type 2 diabetes, glycogen storage disease, galactosemia, or cancer. In such embodiments, the cancer can be selected from the group consisting of acute myelogenous leukemia (AML), chronic myelogenous leukemia (CML), acute lymphocytic leukemia (ALL), chronic lymphocytic leukemia (CLL), prolymphocytic leukemia (PLL), large granular lymphocytic (LGL) leukemia, and hairy cell leukemia (HCL). In some such embodiments, the cancer can be a non-Hodgkin lymphoma or a Hodgkin's lymphoma. In such embodiments, the cancer can be selected from the group consisting of follicular lymphoma, Burkitt lymphoma, Waldenström macroglobulinemia, diffuse large B cell lymphoma, primary mediastinal B cell lymphoma, small lymphocytic lymphoma, marginal zone lymphoma, mantle cell lymphoma, peripheral T cell lymphoma (not otherwise specified), anaplastic large cell lymphoma, angioimmunoblastic lymphoma, and cutaneous T cell lymphoma. In other such embodiments, the cancer can be selected from the group consisting of nodular sclerosis Hodgkin lymphoma, mixed cellularity Hodgkin lymphoma, lymphocyte-rich Hodgkin's disease, and lymphocyte-depleted Hodgkin's disease. In other such embodiments, the cancer can be selected from the group consisting of basal cell carcinoma, squamous cell carcinoma, melanoma, cutaneous T-cell lymphoma, dermatofibrosarcoma protuberans, Merkel cell carcinoma, and sebaceous carcinoma. In some embodiments, the method can predict, diagnose, or monitor a dermatological disease or disorder in a subject in need thereof. In such embodiments, the dermatological disease or disorder can be psoriasis, acne vulgaris, hidradenitis suppurativa, androgenic alopecia, acanthosis nigricans, or atopic dermatitis.

Methods of measuring the degree of cellular energy metabolism can predict, diagnose, or monitor a pathological condition in a subject in need thereof. In such embodiments, the pathological condition can be acral dry gangrene, carotenosis, diabetic dermopathy, diabetic bulla, diabetic cheiroarthropathy, malum perforans, necrobiosis lipoidica, scleredema, waxy skin, diabetic foot, diabetic foot ulcer, or neuropathic arthropathy.

Methods of measuring the degree of cellular energy metabolism can be used on any bodily surface comprising cells. In some embodiments, the bodily surface comprising cells can be a mucosal or dermal surface of a subject. In such embodiments, the bodily surface comprising cells can be an epidermis, a dermis, a subcutaneous tissue, or a combination thereof of a dermal surface of a subject. In other such embodiments, the bodily surface comprising cells can be an epithelial layer, a lamina propria, a muscularis mucosa, a submucosal layer, a muscle, or a combination thereof of a mucosal surface of a subject.

Methods of measuring the degree of cellular energy metabolism can be used on any solution comprising cells. In some embodiments, the solution comprising cells can be a cell culture. In such embodiments, the cell culture can be a biopsy explant.

### BRIEF DESCRIPTION OF THE DRAWINGS

FIGS. 1A-1E are a series of schematics for the sensor substrate design of cellular energy metabolism sensor devices with one bar shaped transmitting plane. Dark gray is the ground plane, light gray is the transmitting plane, white is a gap between the ground plane and transmitting plane. Transmitting plane width is labeled in millimeters (mm) where applicable. FIG. 1A is a sensor substrate schematic of a sensor device with one 6 mm (width) transmitting plane. FIG. 1B is a sensor substrate schematic of a sensor device with one 5 mm (width) transmitting plane. FIG. 1C is a sensor substrate schematic of a sensor device with one 4 mm (width) transmitting plane. FIG. 1D is a sensor substrate schematic of a sensor device with one 3 mm (width) transmitting plane. FIG. 1E is a sensor substrate schematic of a sensor device with one 2 mm (width) transmitting plane.

FIGS. 2A-2E are a series of schematics for the sensor substrate design of cellular energy metabolism sensor devices with two transmitting planes or other transmitting plane geometries. Dark gray is the ground plane, light gray is the transmitting plane(s), white is a gap between the ground plane and transmitting plane. Transmitting plane width is labeled in millimeters (mm) where applicable. FIG. 2A is a sensor substrate schematic of a sensor device with a 4 mm (width) transmitting plane and another smaller transmitting plane. FIG. 2B is a sensor substrate schematic of a sensor device with a 4 mm (width) transmitting plane and another smaller transmitting plane. FIG. 2C is a sensor substrate schematic of a sensor device with a 2 mm (width) transmitting plane and another smaller transmitting plane. FIG. 2D is a sensor substrate schematic of a sensor device with two 2 mm (width) transmitting planes. FIG. 2E is a sensor substrate schematic of a sensor device with one annular transmitting plane.

FIGS. 3A and 3B are graphs showing experimental in vivo detection using LIBRE (an interstitial glucose sensor, ABBOTT LABORATORIES) and the cellular energy metabolism monitoring device taped to a subject. The subject consumed glucose serially at three separate timepoints. FIG. 3A is a graph of the interstitial glucose levels (mg/dl) over the time course of the three glucose feedings. FIG. 3B is a graph of a cellular energy metabolism sensor device reading (measured in impedance by a change in magnitude at 64 MHz) over the course of the same experiment. The x-axes of both FIG. 3A and FIG. 3B are aligned to the same time. While the LIBRE® shows three separate glucose excursion events, the cellular energy metabolism sensor device shows a signal occurring only during the final excursion event. This observation is believed to indicate that cellular energy metabolism is only changing in response to the final glucose consumption.

FIG. 4 is a graph showing experimental in vivo detection of a glucose excursion by a LIBRE® device. A subject was monitored by taped LIBRE® device for glucose levels after consuming a glucose snack. The x axis is time, and the y-axis is LIBRE® device measurement in (mg/dL). The LIBRES device shows a steep glucose excursion. This glucose measurement corresponds to the same experiment as shown in FIG. 5.

FIG. 5 is a graph showing experimental in vivo detection of a glucose excursion by a cellular energy metabolism monitoring device of this disclosure. The x axis is time, and the y-axis is a cellular energy metabolism monitoring device measurement of the change in magnitude at 64 MHz. This metabolic measurement corresponds to the same experiment as shown in FIG. 4. The same subject also had a cellular energy metabolism monitoring device taped on to their skin during the course of the experiment. In this experiment, the sensor device produced a signal peak overlapping with the glucose measurement, but the decline in signal was more gradual than the decline observed in FIG. 4 for interstitial glucose measurement. Without wishing to be bound by any one particular mechanism or theory, it is believed the gradual peak shape of the cellular energy metabolism device reflects the gradual degree of cellular energy metabolism (e.g., cellular ATP production) in response to elevated glucose concentrations.

FIG. 6 is a graph of an in vitro experiment measuring the degree of cellular energy metabolism of a cell suspension with a cellular energy metabolism monitoring device. Three solutions of Jurkat T cells were starved in low-growth medium. At approximately 21 minutes (0:21), a solution of D-glucose in culture medium was added to create a 200 mg/dl D-glucose solution. This resulted in an observed signal peak. In contrast, addition of either medium with low glucose or medium with non-metabolized 2-deoxy-D-glucose resulted in no observable peak with the cellular energy metabolism monitoring device. The y-axis of each curve is shifted to normalize all baseline values before glucose or control addition.

FIG. 7 is a graph of an in vitro experiment measuring the intracellular production of adenosine triphosphate (ATP). Two solutions of Jurkat T cells were stimulated either with Dulbecco's Modified Eagle Medium (DMEM) with 180 mg/dl glucose added to ‘plain medium’ (DMEM, low glucose, 5.5 mM, 100 mg/dL) or ‘plain medium’ alone. The y-axis is luciferase activity in relative luminescence units (RLUs) and the x-axis is time after stimulation in minutes. Error bars indicate an average from triplicate measurements. Following stimulation (at time 0), intracellular ATP concentrations were measured over the course of the experiment using a luciferase-based assay. The graph shows that stimulation with glucose results in an initial increase and a slower decrease in intracellular ATP concentrations as measured by luciferase activity.

FIG. 8 is a graph of the difference in intracellular ATP between media stimulation with high or low glucose. The graph is generated from calculation of the difference (delta) in luciferase activity between DMEM stimulation with high or low glucose (RLU with added glucose (‘plane medium’ plus 180 mg/dl glucose)-without added glucose (‘plain medium’)) over time (minutes following stimulation) at each time point from the experiment shown in FIG. 7. The curve shows an increase in the difference of intracellular ATP production (as determined by luciferase activity) over 1-2 hours followed by a decrease in the difference over about 6 hours.

FIGS. 9A-9D are graphs of the degree of cellular energy metabolism in Jurkat T cells following various stimuli by observation of radio frequency magnitude. The x-axis is time of day (hour: minute) and the y-axis is radio frequency magnitude (mV, after linear temperature correction). FIG. 9A is a graph of radio frequency magnitude following treatment of Jurkat T cells with D-glucose (180 mg/dl final concentration) in DMEM. FIG. 9B is a graph of radio frequency magnitude following treatment with 2-deoxy-D-glucose (2DG, 180 mg/dl) in DMEM. FIG. 9C is a graph of radio frequency magnitude following treatment with D-glucose (180 mg/dl final concentration) and BAY-876 (5 μM final concentration, an inhibitor of glucose transporter 1) in DMEM. FIG. 9D is a graph is a graph of radio frequency magnitude following treatment with DMEM with low D-glucose (5.5 mM, 100 mg/dl, ‘plain medium’). The decrease in radio frequency magnitude (the negative slope) is due to consumption of ATP over time. While plain medium (FIG. 9D) and BAY-876 with D-glucose (FIG. 9C) stimuli show linear plots after temperature re-equilibration, 2DG (FIG. 9B) and D-glucose (FIG. 9A) stimuli show initial peaks in radio frequency magnitude. D-glucose stimulation (FIG. 9A) additionally shows a slower, less linear decline in radio frequency magnitude than 2DG when compared to ‘plain medium’.

FIG. 10 is a graph of the extracellular glucose concentration. The experiment is the identical to that shown in FIG. 9A. D-glucose (mg/dl) was measured as a function of time (hours: minutes), following stimulation with D-glucose at time point 0:00. 6 hours following the stimulation with glucose, the extracellular glucose concentration approaches 0 mg/dl.

FIGS. 11A-11C are graphs of the difference (delta or delta, delta) in radio frequency magnitude with stimuli (D-glucose or 2DG) versus DMEM with low D-glucose (5.5 mM, 100 mg/dl, ‘plain medium’) from the same experiment and data as FIG. 9A, FIG. 9B, and FIG. 9D. FIG. 11A is a graph of the difference (delta) in radio frequency magnitude with D-glucose stimulus between FIG. 9A (D-glucose 180 mg/dl final concentration in DMEM) and FIG. 9D (DMEM with low D-glucose (5.5 mM, 100 mg/dl), ‘plain medium’). FIG. 11B is a graph showing the difference (delta) in radio frequency magnitude with 2-deoxy-D-glucose stimulus between FIG. 9B (2-deoxy-D-glucose 180 mg/dl final concentration in DMEM) and FIG. 9D (DMEM with low D-glucose (5.5 mM, 100 mg/dl), ‘plain medium’). FIG. 11C is a graph showing the difference (delta, delta) in radio frequency magnitude in D-glucose and 2-deoxy-D-glucose stimuli between FIG. 11A and FIG. 11B. The corresponding graph closely mimics (slowly increasing and decreasing over about 6 hours) the shape of that observed for intracellular ATP as shown in FIG. 8, indicating that radio frequency magnitude changes reflect changes in intracellular ATP levels and production due to cellular energy metabolism.

### DETAILED DESCRIPTION

Current metabolic assays measuring the degree of cellular energy metabolism (e.g., glucose consumption to form ATP) are limited to complicated instruments and techniques. For example, Clark electrodes are used to measure oxygen consumption (see Winqvist, I. et al. Altered density, metabolism and surface receptors of eosinophils in eosinophilia (1982) Immunology 47, 531-539). Transcriptomic or proteomic approaches can measure gene and protein expression as a secondary measure of cellular energy metabolism. Additionally, radiometric or luminescent assays employing detectable substrates or products have been employed in vitro (see Csepregi R. et al. A One-Step Extraction and Luminescence Assay for Quantifying Glucose and ATP Levels in Cultured HepG2 Cells (2018) Int. J. Mol. Sci. 19, 9, 2670). More recent technology utilizes sensor arrays to measure extracellular changes in oxygen consumption and extracellular acidification, which allows for a more rapid analysis, but at a high instrument cost and only applicable to in vitro analyses (see Zhang J. and Zhang Q., Using Seahorse Machine to Measure OCR and ECAR in Cancer Cells (2019), Cancer Metabolism: Methods and Protocols, Methods in Molecular Biology, 1928, 353-363).

This disclosure relates to non-invasive methods of measuring and monitoring the degree of cellular energy metabolism in vitro or in vivo. Methods of this disclosure allow for the measurement of the degree of cellular energy metabolism without the need for invasive needles or lancets that puncture the skin and increase the risk for infection. Moreover, in vitro measurement of the degree of cellular energy metabolism is simplified by use of a single device and measurement compatible with any cell suspension or cell culture. It was discovered that by measuring the impedance (e.g., resistance) of a radio frequency in a circuit containing a transmitting plane (e.g., a component comprising an antenna) in contact with (e.g., impinging on) a biological matrix containing cells (e.g., a bodily surface, a mucosal surface, a dermal surface, a solution comprising cells), the resulting change in impedance (e.g., bioimpedance, resistance, capacitance) is correlated with the degree of metabolism of the cells. Without wishing to be bound by any one particular mechanism of operation or theory, it is believed that the active consumption of an energy source (e.g., glucose) and production of adenosine triphosphate (ATP) by cells within the electric field of a cellular energy metabolism sensor device results in an increase in radio frequency impedance (e.g., resistance) in the device. It is further believed that, by transmitting a signal at a suitable frequency, the impedance measured by the circuitry of the cellular energy metabolism sensor device can be correlated to the degree of energy source (e.g., glucose) consumption and ATP production. This impedance measurement can further be correlated to physiological measurements such as cell vitality, cell respiration, and the degree of cellular energy metabolism.

This disclosure relates to methods of non-invasively measuring the degree of cellular energy metabolism by contacting (e.g., adhering, creating an interface with, touching, pressing, dipping, submerging) a monitoring device for detecting cellular energy metabolism (e.g., a cellular energy metabolism sensor device, a sensor device, a cellular energy metabolism monitoring device, a monitoring device) with a bodily surface or a solution comprising cells.

Methods can comprise generating a transmitted signal in a cellular energy metabolism sensor device in contact with a bodily surface or solution comprising cells.

Measuring one or more operating values of the circuitry in methods disclosed herein can determine impedance, voltage, current (e.g., bioimpedance, resistance, capacitance) of the radio frequency of a transmitted signal of a sensor substrate in contact with a bodily surface or a solution comprising cells.

In methods of this disclosure, the monitoring device is maintained in contact with a bodily surface or a solution comprising cells for a desired length of time (e.g., any length of time).

Methods can further comprise collecting data from the sensor substrate of the monitoring device (e.g., impedance, bioimpedance, resistance, capacitance, voltage, current data) that indicates (e.g., is correlated to, is related to) the change in the degree of cellular energy metabolism for the bodily surface or solution comprising cells. For example, methods can determine the energy metabolism for a desired length of time (e.g., any length of time).

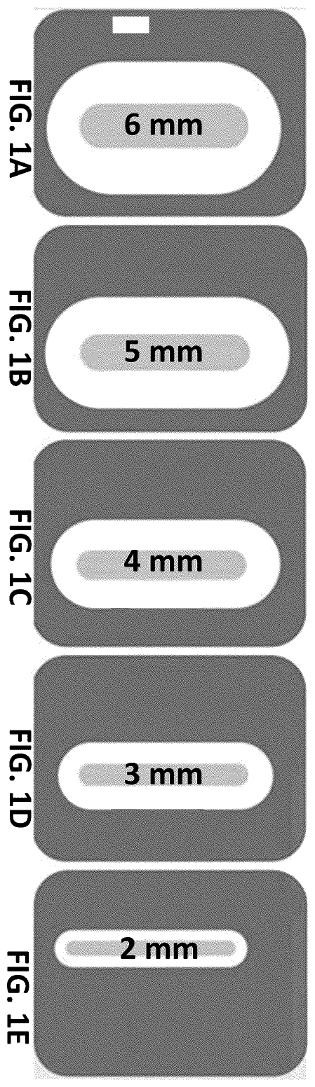

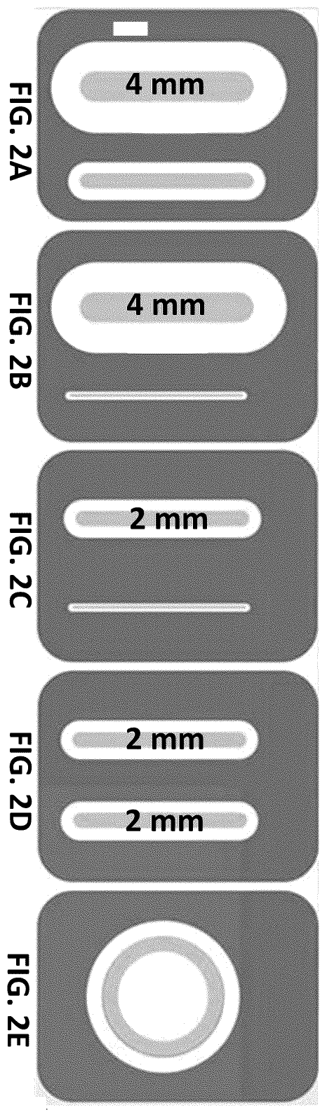

Methods of this disclosure utilize a monitoring device for detecting the degree of cellular energy metabolism in a bodily surface or solution comprising cells. Monitoring devices can contain sensor substrates that can comprise any number of transmitting planes (e.g., one or more transmitting planes) and a ground plane. The transmitting planes can transmit a transmitted signal at a radio frequency. Measuring the transmitted signal magnitude and/or phase of the circuit or other operating values of the circuit (e.g., resistance, impedance, electrical potential, current, capacitance) as a function of cell solution or tissue loads within the electric field of the transmitting plane indicates the degree of cellular energy metabolism (e.g., production of ATP) in the surrounding cell solution or tissue. Specifically, change in the magnitude and/or phase of the transmitted signals (e.g., radio frequencies) of the circuit at a particular frequency can be used to calculate operating values of the circuit (e.g., bioimpedance, resistance, capacitance, reactance), which can be correlated to the degree of cellular energy metabolism (e.g., glucose consumption, ATP production). The shape of the ground plane of the device and the gap distance between the transmitting plane(s) and the ground plane impact the penetration depth of the electric field into the skin. By changing the geometry of the transmitting and ground planes, as well as the gap between the planes, the depth of detection and sensitivity to the degree of cellular energy metabolism (e.g., changes in ATP concentration) on a bodily surface or a solution comprising cells can be controlled. Exemplary sensor substrate structure shapes are shown in FIGS. 1A-1E and FIGS. 2A-2E.

In some embodiments, the method of non-invasive measuring the degree of cellular energy metabolism in a bodily surface or a solution comprising cell can comprise the steps of
a) contacting a monitoring device for detecting the degree of cellular energy metabolism to a bodily surface or a solution comprising cells,
b) generating a transmitted signal at a radio frequency,
c) measuring one or more operating values of the circuitry of the monitoring device at the radio frequency of the transmitted signal in contact with the bodily surface or the solution comprising cells,
d) maintaining contact with the bodily surface or the solution comprising cells for a desired length of time, and
e) collecting impedance data, voltage data, or current data from the sensor substrate of the monitoring device that indicates the degree of cellular energy metabolism for the bodily surface or the solution comprising cells.

### Radio Frequency

The radio frequency of the transmitted signal can be any frequency. Without wishing to be bound to any one particular mechanism of operation or theory, it is believed that certain radio frequencies of transmitted signal in contact with a bodily surface or a solution comprising cells generate a bioimpedance, voltage, or current, that can be correlated with the degree of cellular energy metabolism (e.g., cellular respiration, production of ATP, consumption of glucose, cell viability). The transmitted signal radio frequency can range from about 0.1 MHz to about 250 MHz, e.g., about 0.1 MHz to about 200 MHz, about 0.1 MHz to about 160 MHz, about 0.1 MHz to about 130 MHz, about 0.1 MHz to about 110 MHz, about 0.1 MHz to about 90 MHz, about 0.1 MHz to about 80 MHz, about 0.1 MHz to about 70 MHz, about 0.1 MHz to about 65 MHz, about 0.1 MHz to about 60 MHz, about 0.1 MHz to about 55 MHz, about 0.1 MHz to about 50 MHz, about 0.1 MHz to about 45 MHz, about 0.1 MHz to about 40 MHz, about 0.1 MHz to about 35 MHz, about 0.1 MHz to about 30 MHz, about 0.1 MHz to about 25 MHz, about 0.1 MHz to about 20 MHz, about 0.1 MHz to about 15 MHz, about 0.1 MHz to about 12 MHz, about 0.1 MHz to about 9 MHz, about 0.1 MHz to about 8 MHz, about 0.1 MHz to about 7 MHz, about 0.1 MHz to about 6 MHz, about 0.1 MHz to about 5 MHz, about 0.1 MHz to about 4 MHz, about 0.1 MHz to about 3 MHz, about 0.1 MHz to about 2 MHz, about 0.1 MHz to about 1 MHz, about 0.1 MHz to about 0.5 MHz, about 0.5 MHz to about 200 MHz, about 0.5 MHz to about 160 MHz, about 0.5 MHz to about 130 MHz, about 0.5 MHz to about 110 MHz, about 0.5 MHz to about 90 MHz, about 0.5 MHz to about 80 MHz, about 0.5 MHz to about 70 MHz, about 0.5 MHz to about 65 MHz, about 0.5 MHz to about 60 MHz, about 0.5 MHz to about 55 MHz, about 0.5 MHz to about 50 MHz, about 0.5 MHz to about 45 MHz, about 0.5 MHz to about 40 MHz, about 0.5 MHz to about 35 MHz, about 0.5 MHz to about 30 MHz, about 0.5 MHz to about 25 MHz, about 0.5 MHz to about 20 MHz, about 0.5 MHz to about 15 MHz, about 0.5 MHz to about 12 MHz, about 0.5 MHz to about 9 MHz, about 0.5 MHz to about 8 MHz, about 0.5 MHz to about 7 MHz, about 0.5 MHz to about 6 MHz, about 0.5 MHz to about 5 MHz, about 0.5 MHz to about 4 MHz, about 0.5 MHz to about 3 MHz, about 0.5 MHz to about 2 MHz, about 0.5 MHz to about 1 MHz, about 1 MHz to about 250 MHz, about 2 MHz to about 250 MHz, about 3 MHz to about 250 MHz, about 4 MHz to about 250 MHz, about 5 MHz to about 250 MHz, about 6 MHz to about 250 MHz, about 7 MHz to about 250 MHz, about 8 MHz to about 250 MHz, about 9 MHz to about 250 MHz, about 10 MHz to about 250 MHz, about 11 MHz to about 250 MHz, about 12 MHz to about 250 MHz, about 15 MHz to about 250 MHz, about 20 MHz to about 250 MHz, about 25 MHz to about 250 MHz, about 30 MHz to about 250 MHz, about 35 MHz to about 250 MHz, about 40 MHz to about 250 MHz, about 45 MHz to about 250 MHz, about 50 MHz to about 250 MHz, about 60 MHz to about 250 MHz, about 70 MHz to about 250 MHz, about 80 MHz to about 250 MHz, about 90 MHz to about 250 MHz, about 100 MHz to about 250 MHz, about 120 MHz to about 250 MHz, about 140 MHz to about 250 MHz, about 160 MHz to about 250 MHz, about 180 MHz to about 250 MHz, about 200 MHz to about 250 MHz, about 225 MHz to about 250 MHz, about 60 MHz to about 68 MHz, about 55 MHz to about 70 MHz, about 40 MHz to about 75 MHz, about 25 MHz to about 75 MHz, about 25 MHz to about 90 MHz, about 15 MHz to about 120 MHz, or about 40 MHz to about 100 MHz. In some embodiments, the transmitted radio frequency is about 49 MHz. In some embodiments, the transmitted radio frequency is about 54 MHz. In some embodiments, the transmitted radio frequency is about 59 MHz. In some embodiments, the transmitted radio frequency is about 64 MHz. In some embodiments the transmitted radio frequency is about 69 MHz. In some embodiments the transmitted radio frequency is about 74 MHz. In some embodiments the transmitted radio frequency is about 79 MHz. In some embodiments the transmitted radio frequency is about 84 MHz. In some embodiments, the transmitted radio frequency ranges from about 0.5 to about 250 MHz. In some embodiments, the transmitted radio frequency ranges from about 40 to about 75 MHz.

### Transmitting Plane

The transmitting plane in devices described herein can contain one or more antennas that generate an electric field. Bodily surfaces or solutions comprising cells within the electric field can change the operating values (e.g., resistance, impedance) of the circuitry of the cellular energy metabolism sensor device. It is believed, without wishing to be bound by any one particular mechanism or theory, that consumption of an energy source (e.g., glucose) and production of ATP can change the degree of impedance (e.g., bioimpedance) measured by the device. The monitoring device for measuring the degree of cellular energy metabolism (e.g., a sensor substrate of monitoring device) can contain any number of transmitting planes. The number of transmitting planes in the monitoring device can be one or more, e.g., one transmitting plane, two transmitting planes, three transmitting planes, four transmitting planes, five transmitting planes, six transmitting planes, seven transmitting planes, eight transmitting planes, nine transmitting planes, ten transmitting planes, or more transmitting planes. In some embodiments, the device comprises one or more transmitting planes. In some embodiments, the device comprises one transmitting plane. In some embodiments, the device comprises two transmitting planes. In some embodiments, the device comprises three transmitting planes. In some embodiments, the device comprises four transmitting planes.

The transmitting plane can be any shape. The transmitting plane can be bar shaped. The transmitting plane can be rectangular shaped. The transmitting plane can be square shaped. The transmitting plane can be circular shaped. The transmitting plane can be pentagonal. The transmitting plane can be hexagonal. The transmitting plane can be annular (e.g., ring shaped). The transmitting plane can be triangular shaped. In some embodiments, the one or more of the transmitting planes is annular. In some embodiments, one or more of the transmitting planes is bar shaped. In such embodiments, the bar shaped transmitting plane can have any width. In such embodiments, the transmitting plane can have a width ranging from about 0.25 mm to about 15 mm, e.g., about 0.25 mm to about 12 mm, about 0.25 mm to about 10 mm, about 0.25 mm to about 9 mm, about 0.25 mm to about 8 mm, about 0.25 mm to about 7 mm, about 0.25 mm to about 6 mm, about 0.25 mm to about 5 mm, about 0.25 mm to about 4.5 mm, about 0.25 mm to about 4 mm, about 0.25 mm to about 3.5 mm, about 0.25 mm to about 3 mm, about 0.25 mm to about 2.75 mm, about 0.25 mm to about 2.5 mm, about 0.25 mm to about 2.25 mm, about 0.25 mm to about 2 mm, about 0.25 mm to about 1.75 mm, about 0.25 mm to about 1.5 mm, about 0.25 mm to about 1.25 mm, about 0.25 mm to about 1.0 mm, about 0.25 mm to about 0.75 mm, about 0.25 mm to about 0.5 mm, about 0.5 mm to about 15 mm, about 0.75 mm to about 15 mm, about 1 mm to about 15 mm, about 1.25 mm to about 15 mm, about 1.5 mm to about 15 mm, about 1.75 mm to about 15 mm, about 2 mm to about 15 mm, about 2.25 mm to about 15 mm, about 2.5 mm to about 15 mm, about 2.75 mm to about 15 mm, about 3 mm to about 15 mm, about 3.5 mm to about 15 mm, about 4 mm to about 15 mm, about 4.5 mm to about 15 mm, about 5 mm to about 15 mm, about 5.5 mm to about 15 mm, about 6 mm to about 15 mm, about 6.5 mm to about 15 mm, about 7 mm to about 15 mm, about 8 mm to about 15 mm, about 9 mm to about 15 mm, about 10 mm to about 15 mm, about 11 mm to about 15 mm, about 12 mm to about 15 mm, about 14 mm to about 15 mm, about 1 mm to about 8 mm, about 1 mm to about 7 mm, about 0.5 mm to about 9 mm, about 1 mm to about 9 mm, or about 1 mm to about 10 mm. In some embodiments, the bar shaped transmitting plane has a width of about 1 mm. In some embodiments, the bar shaped transmitting plane has a width of about 2 mm. In some embodiments, the bar shaped transmitting plane has a width of about 3 mm. In some embodiments, the bar shaped transmitting plane has a width of about 4 mm. In some embodiments, the bar shaped transmitting plane has a width of about 5 mm. In some embodiments, the bar shaped transmitting plane has a width of about 6 mm. In some embodiments, the bar shaped transmitting plane has a width of about 7 mm. In some embodiments, the bar shaped transmitting plane has a width of about 8 mm. In some embodiments, the bar shaped transmitting plane has a width of about 1 mm to about 8 mm. In some embodiments, the bar shaped transmitting plane has a width of about 0.25 mm to about 15 mm.

The transmitting plane can have any width. The transmitting plane can have a width ranging from about 0.25 mm to about 15 mm, e.g., about 0.25 mm to about 12 mm, about 0.25 mm to about 10 mm, about 0.25 mm to about 9 mm, about 0.25 mm to about 8 mm, about 0.25 mm to about 7 mm, about 0.25 mm to about 6 mm, about 0.25 mm to about 5 mm, about 0.25 mm to about 4.5 mm, about 0.25 mm to about 4 mm, about 0.25 mm to about 3.5 mm, about 0.25 mm to about 3 mm, about 0.25 mm to about 2.75 mm, about 0.25 mm to about 2.5 mm, about 0.25 mm to about 2.25 mm, about 0.25 mm to about 2 mm, about 0.25 mm to about 1.75 mm, about 0.25 mm to about 1.5 mm, about 0.25 mm to about 1.25 mm, about 0.25 mm to about 0.75 mm, about 0.25 mm to about 0.5 mm, about 0.5 mm to about 15 mm, about 0.75 mm to about 15 mm, about 1 mm to about 15 mm, about 1.25 mm to about 15 mm, about 1.5 mm to about 15 mm, about 1.75 mm to about 15 mm, about 2 mm to about 15 mm, about 2.25 mm to about 15 mm, about 2.5 mm to about 15 mm, about 2.75 mm to about 15 mm, about 3 mm to about 15 mm, about 3.5 mm to about 15 mm, about 4 mm to about 15 mm, about 4.5 mm to about 15 mm, about 5 mm to about 15 mm, about 5.5 mm to about 15 mm, about 6 mm to about 15 mm, about 6.5 mm to about 15 mm, about 7 mm to about 15 mm, about 8 mm to about 15 mm, about 9 mm to about 15 mm, about 10 mm to about 15 mm, about 11 mm to about 15 mm, about 12 mm to about 15 mm, about 14 mm to about 15 mm, about 1 mm to about 8 mm, about 1 mm to about 7 mm, about 0.5 mm to about 9 mm, about 1 mm to about 9 mm, or about 1 mm to about 10 mm. In some embodiments, the bar shaped transmitting plane has a width of about 1 mm. In some embodiments, the transmitting plane has a width of about 2 mm. In some embodiments, the transmitting plane has a width of about 3 mm. In some embodiments, the transmitting plane has a width of about 4 mm. In some embodiments, the transmitting plane has a width of about 5 mm. In some embodiments, the transmitting plane has a width of about 6 mm. In some embodiments, the transmitting plane has a width of about 7 mm. In some embodiments, transmitting plane has a width of about 8 mm. In some embodiments, the transmitting plane has a width of about 1 mm to about 8 mm. In some embodiments, the transmitting plane has a width of about 0.25 mm to about 15 mm.

### Ground Plane

Monitoring devices (e.g., sensor substrates of monitoring devices) for cellular energy metabolism used in methods of this disclosure can comprise a ground plane. Both the shape of the ground planes and the gap distance between the transmitting planes and ground planes of this disclosure can impact (e.g., alter, adjust, control) the penetration depth of the electric field of a transmitted signal (e.g., radio signal, radio frequency) and the sensitivity of detection of ATP changes by the cellular energy metabolism sensor device on a bodily surface or a solution comprising cells for impedance (e.g., bioimpedance, resistance) measurement. Monitoring devices can comprise ground planes of any shape or size. Monitoring devices can comprise one or more ground planes. Monitoring devices can comprise two or more ground planes. Monitoring devices can comprise three or more ground planes. Monitoring devices can comprise four or more ground planes. Monitoring devices can comprise five or more ground planes. In some embodiments, monitoring devices can comprise one ground plane. In some embodiments, monitoring devices can comprise two ground planes. In some embodiments, monitoring devices can comprise three ground planes. In some embodiments, monitoring devices can comprise four ground planes. In some embodiments, monitoring devices can comprise five ground planes. In some embodiments, monitoring devices can comprise six ground planes. In some embodiments, monitoring devices can comprise seven ground planes. In some embodiments, monitoring devices can comprise eight ground planes. In some embodiments, monitoring devices can comprise nine ground planes. In some embodiments, monitoring devices can comprise ten ground planes.

### Bodily Surfaces

Methods of this disclosure can measure the degree of the cellular energy metabolism of any bodily surface (e.g., a bodily surface comprising cells). The bodily surface can be an exterior bodily surface (e.g., a dermal surface, the skin). In some embodiments, the bodily surface is a dermal surface. Dermal surfaces for measuring the degree of cellular energy metabolism can include skin of any location including, but not limited to, a hand (e.g., palm, finger), arm (e.g., wrist, forearm, shoulder, elbow, armpit), chest, abdomen, back, neck, buttocks, leg (e.g., thigh, calf, shin, ankle, knee), foot (e.g., footpad, toe), head (e.g., ear, face, scalp, nose), and combinations thereof. In some embodiments, the method measures the degree of the cellular energy metabolism of a dermal surface on a forearm. In some embodiments, the method measures the degree of the cellular energy metabolism of a dermal surface on an upper arm. In some embodiments, the method measures the degree of the cellular energy metabolism of a dermal surface on a wrist. In some embodiments, the method measures the degree of the cellular energy metabolism of a dermal surface on a palm. In some embodiments, the method measures the degree of the cellular energy metabolism of a dermal surface on a finger.

The methods of this disclosure can measure the degree of the cellular energy metabolism of any layer of the dermal surface. The degree of cellular energy metabolism of any dermal surface layer can be measured including, but not limited to, an epidermis, a dermis, a subcutaneous tissue, or a combination thereof of a dermal surface of a subject. In some embodiments, the method measures the degree of the cellular energy metabolism of an epidermis. In some embodiments, the method measures the degree of the cellular energy metabolism of a dermis. In some embodiments, the method measures the degree of the cellular energy metabolism of a subcutaneous tissue.

Methods of this disclosure can measure the degree of the cellular energy metabolism of a mucosal surface (e.g., a mucosal surface comprising cells) of a subject. The mucosal surface can be any surface of a subject covered by epithelium that secretes or is covered with mucus, including but not limited to, aural mucosa (e.g., middle ear), oral mucosa (e.g., frenulum), esophageal mucosa, rectal mucosa, gastric mucosa, intestinal mucosa (e.g., rectal mucosa, small intestinal mucosal, large intestinal mucosa), respiratory mucosa, vaginal mucosa, urethral mucosa, endometrial mucosa, nasal mucosa (e.g., olfactory mucosa), penile mucosa, conjunctiva mucosa, and combinations thereof. This disclosure relates to methods of measuring the degree of cellular energy metabolism in any layer (e.g., portion, segment, cellular component) of a mucosal surface including, but not limited to, an epithelial layer, a lamina propria, a muscularis mucosa, a submucosal layer, a muscle, or a combination thereof. In some embodiments, the bodily surface comprising cells is an epithelial layer, a lamina propria, a muscularis mucosa, a submucosal layer, a muscle, or a combination thereof of a mucosal surface of a subject. In some embodiments, the bodily surface comprising cells is a mucosal or dermal surface of a subject.

### Solutions Comprising Cells

Methods of this disclosure can measure the degree of the cellular energy metabolism of any solution comprising cells. The solution comprising cells can be a cell culture. The cell culture can be a biopsy explant (e.g., a cell culture from cells from a biopsy of a subject in need thereof). The cell culture or solution comprising cells can be any variety of cells including, but not limited to, primary cells from a subject or immortalized cells (e.g., cancer cells). In some embodiments, the cell culture comprises immortalized cells. In some embodiments, the cell culture comprises cancer cells. Cells can be from any animal (e.g., a human, a mouse, a dog, a cat, a horse, a goat, a llama, a camel, a fish (e.g., a shark), an amphibian, a bird). Cells can be of any differentiation state including, but not limited to, stem cells (e.g., totipotent stem cells, pluripotent stem cells, unipotent stem cells) or differentiated cells. Cells can be of any cell lineage including, but not limited to, lymphoid cells (T cells (e.g., cytotoxic T cells, CD8+ T cells, CD4+T cells, memory T cells, naïve T cells, regulatory T cells), B cells (e.g., plasma cells, memory B cells, naïve B cells, a hybridoma, plasmablast cells, regulatory B cells), and natural killer cells), myeloid cells (e.g., macrophages, neutrophils, basophils, monocytes, eosinophils, mast cells, megakaryocytes), neuronal cells (e.g., myelin sheath), dermal cells (e.g., epidermal cells, epithelial cells, melanocytes), mucosal cells (e.g., lamina propria cells, muscularis cells, epithelium), red blood cells, and bone marrow cells. Cells can be from any organ or bodily locale including, but not limited to, the adrenal gland, the bile duct, the blood vessel, the bone, the bone marrow, the brain, the cartilage, the eye, the fat, the gallbladder, the gastrointestinal tract (e.g., the large intestines, the mouth, the rectum, the small intestines, the stomach, the stroma, the throat), the heart, the kidney, the ligament, the liver, the lung, the lymph node, the mouth, the muscle, the ovary, the pancreas, the skin, and the testis. In some embodiments, the solution comprising cells is a cell culture. In some such embodiments, the cell culture can be a biopsy explant. In some embodiments, the cell culture comprises Jurkat T cells.

### Contact Time

This disclosure relates to methods of measuring the degree of cellular energy metabolism by detecting one or more operating values (e.g., impedance, bioimpedance, resistance, capacitance of a radio signal) in a device after transmitting a radio frequency signal that corresponds to the degree of cellular energy metabolism for a desired length of time. In methods of this disclosure, the monitoring device for detecting cellular energy metabolism can be held in contact (e.g., can maintain contact, press) with a bodily surface or solution containing cells for any desired length of time. Contact can be made with a surface or solution ranging from about 1 second to about 24 hours, e.g., about 5 seconds to about 24 hours, about 10 seconds to about 24 hours, about 15 seconds to about 24 hours, about 20 seconds to about 24 hours, about 25 seconds to about 24 hours, about 30 seconds to about 24 hours, about 40 seconds to about 24 hours, about 45 seconds to about 24 hours, about 60 seconds (e.g., 1 minute) to about 24 hours, about 1.5 minutes to about 24 hours, about 2 minutes to about 24 hours, about 2.5 minutes to about 24 hours, about 3 minutes to about 24 hours, about 3.5 minutes to about 24 hours, about 4 minutes to about 24 hours, about 5 minutes to about 24 hours, about 6 minutes to about 24 hours, about 7 minutes to about 24 hours, about 8 minutes to about 24 hours, about 9 minutes to about 24 hours, about 10 minutes to about 24 hours, about 12 minutes to about 24 hours, about 14 minutes to about 24 hours, about 16 minutes to about 24 hours, about 18 minutes to about 24 hours, about 20 minutes to about 24 hours, about 25 minutes to about 24 hours, about 30 minutes to about 24 hours, about 35 minutes to about 24 hours, about 40 minutes to about 24 hours, about 45 minutes to about 24 hours, about 50 minutes to about 24 hours, about 1 hour to about 24 hours, about 2 hours to about 24 hours, about 3 hours to about 24 hours, about 4 hours to about 24 hours, about 5 hours to about 24 hours, about 6 hours to about 24 hours, about 7 hours to about 24 hours, about 8 hours to about 24 hours, about 9 hours to about 24 hours, about 10 hours to about 24 hours, about 12 hours to about 24 hours, about 14 hours to about 24 hours, about 16 hours to about 24 hours, about 18 hours to about 24 hours, about 20 hours to about 24 hours, about 22 hours to about 24 hours, about 1 minute to about 2 minutes, about 1 minute to about 3 minutes, about 1 minute to about 4 minutes, about 1 minute to about 5 minutes, about 1 minute to about 6 minutes, about 1 minute to about 7 minutes, about 1 minute to about 8 minutes, about 1 minute to about 10 minutes, about 1 minute to about 15 minutes, about 1 minute to about 20 minutes, about 1 minute to about 25 minutes, about 1 minute to about 30 minutes, about 1 minute to about 35 minutes, about 1 minute to about 40 minutes, about 1 minute to about 45 minutes, about 1 minute to about 50 minutes, about 1 minute to about 55 minutes, about 1 minute to about 1 hour, about 1 minute to about 2 hours, about 1 minute to about 4 hours, about 1 minute to about 6 hours, about 1 minute to about 8 hours, about 1 minute to about 10 hours, about 1 minute to about 12 hours, about 1 minute to about 16 hours, or about 1 minute to about 20 hours. In some embodiments, the monitoring device is held in contact with a bodily surface or solution comprising cells for a desired length of time ranging from about 1 minute to about 24 hours. In some embodiments, the monitoring device is held in contact with a bodily surface or a solution comprising cells for a desired length of time ranging from about 1 minute to about 2 hours.

### Contacting a Monitoring Device for Measuring the Degree of Cellular Energy Metabolism

In methods of this disclosure, the monitoring device can be contacted (e.g., applied, pressed, touch, placed on an interface) with a bodily surface or a solution comprising cells. The monitoring device can be contacted by any means or method. In some embodiments, the monitoring device can be applied (e.g., contacted, press, touch) to a bodily surface by an attachment selected from the group consisting of a tape, a band, a wrap, an adhesive, and a combination thereof. In some embodiments, the monitoring device can be applied by tape to a bodily surface. In some embodiments, the monitoring device can be applied by a band to a bodily surface. In some embodiments, the monitoring device can be applied by an adhesive to a bodily surface. In some embodiments, the monitoring device can be applied by a wrap to a bodily surface. The monitoring device can be contacted by any means of attachment to a bodily surface. For example, the means of attachment can be by tape, by a band, by a wrap, by an adhesive, or by a combination thereof.

In methods of this disclosure, the monitoring device can be contacted (e.g., dipped, placed at an interface, submerged, wetted) with a solution comprising cells. The monitoring device can be contacted by any means or method. In some embodiments, the monitoring device can be contacted with a solution comprised of cells by dipping, placing at an interface, submerging, or wetting the monitoring device in the solution.

In methods of this disclosure, the sensor substrate of the monitoring device can be separated from the bodily surface or solution comprising cells by an insulative layer. The insulative layer can be made of any material. The insulative layer can be of any thickness. The insulative layer can have a thickness of about 100 μm or less, e.g., the thickness can be about 90 μm or less, about 85 μm or less, about 80 μm or less, about 75 μm or less, about 70 μm or less, about 65 μm or less, about 60 μm or less, about 55 μm or less, about 50 μm or less, about 45 μm or less, about 40 μm or less, about 35 μm or less, about 30 μm or less, about 25 μm or less, about 20 μm or less, about 15 μm or less, about 10 μm or less, about 8 μm or less, about 6 μm or less, about 4 μm or less, about 2 μm or less, about 1 μm or less, about 0.5 μm or less, about 0.25 μm or less, or about 0.1 μm or less. The thickness of the insulative layer can range from about 0.1 μm to about 100 μm, e.g., about 0.1 μm to about 90 μm, about 0.1 μm to about 85 μm, about 0.1 μm to about 80 μm, about 0.1 μm to about 75 μm, about 0.1 μm to about 70 μm, about 0.1 μm to about 65 μm, about 0.1 μm to about 60 μm, about 0.1 μm to about 55 μm, about 0.1 μm to about 50 μm, about 0.1 μm to about 45 μm, about 0.1 μm to about 40 μm, about 0.1 μm to about 35 μm, about 0.1 μm to about 30 μm, about 0.1 μm to about 25 μm, about 0.1 μm to about 20 μm, about 0.1 μm to about 15 μm, about 0.1 μm to about 10 μm, about 0.1 μm to about 8 μm, about 0.1 μm to about 6 μm, about 0.1 μm to about 4 μm, about 0.1 μm to about 2 μm, about 0.1 μm to about 1 μm, about 0.1 μm to about 0.5 μm, about 0.5 μm to about 100 μm, about 1 μm to about 100 μm, about 2 μm to about 100 μm, about 4 μm to about 100 μm, about 6 μm to about 100 μm, about 8 μm to about 100 μm, about 10 μm to about 100 μm, about 15 μm to about 100 μm, about 20 μm to about 100 μm, about 25 μm to about 100 μm, about 30 pm to about 100 μm, about 35 μm to about 100 μm, about 40 μm to about 100 μm, about 45 μm to about 100 μm, about 50 μm to about 100 μm, about 55 μm to about 100 μm, about 60 μm to about 100 μm, about 65 μm to about 100 μm, about 70 μm to about 100 μm, about 75 μm to about 100 μm, about 80 μm to about 100 μm, about 85 μm to about 100 μm, about 90 μm to about 100 μm, about 95 μm to about 100 μm, about 0.2 μm to about 30 μm, about 0.5 μm to about 20 μm, about 1 μm to about 20 μm, about 2 μm to about 18 μm, about 3 μm to about 16 μm, about 4 μm to about 14 μm, about 5 μm to about 12 μm, or about 6 μm to about 10 μm. In some embodiments, the thickness of the insulative layer is about 10 μm or less.

### Prediction, Diagnosis, or Monitor of Diseases, Disorders, or Pathological Conditions

Methods of this disclosure determine (e.g., measure, detect) the degree of cellular energy metabolism that can predict, diagnose, or monitor a disease or disorder in a subject in need thereof. This disclosure relates to methods of predicting, diagnosing, or monitoring any disease or disorder in a subject in need thereof. The methods of this disclosure can be used in predicting, diagnosing, or monitoring the risk of any disease or disorder in a subject thereof. The disease or disorder can be any disease, including but not limited to, a cardiovascular disease (e.g., coronary artery disease, peripheral arterial disease, cerebrovascular disease, renal artery stenosis, aortic aneurysm), a metabolic disease or disorder (e.g., prediabetes, type 1 diabetes, type 2 diabetes, glycogen storage disease, galactosemia, cancer), a dermatological disease or disorder (e.g., psoriasis, acne vulgaris, hidradenitis suppurativa, androgenic alopecia, acanthosis nigricans, or atopic dermatitis), or a combination thereof. In some embodiments, the disease is an infectious disease (e.g., influenza, HIV, polio). The cancer can be any variety of cancer, including but not limited to, basal cell carcinoma, squamous cell carcinoma, melanoma, cutaneous T-cell lymphoma, dermatofibrosarcoma protuberans, Merkel cell carcinoma, and sebaceous carcinoma. In some embodiments, the method can predict, diagnose, or monitor a cardiovascular disease in a subject in need thereof. In some such embodiments, the cardiovascular disease is coronary artery disease, peripheral arterial disease, cerebrovascular disease, renal artery stenosis, or aortic aneurysm. In some embodiments the method predicts, diagnoses, or monitors a metabolic disease or disorder in a subject in need thereof. In some such embodiments, the metabolic disease or disorder is prediabetes, Type 1 diabetes, Type 2 diabetes, glycogen storage disease, galactosemia, or cancer. In some embodiments, the cancer is selected from the group consisting of basal cell carcinoma, squamous cell carcinoma, melanoma, cutaneous T-cell lymphoma, dermatofibrosarcoma protuberans, Merkel cell carcinoma, and sebaceous carcinoma. In some embodiments, the cancer is a leukemia. In some embodiments, the cancer is selected from the group consisting of acute myelogenous leukemia (AML), chronic myelogenous leukemia (CML), acute lymphocytic leukemia (ALL), chronic lymphocytic leukemia (CLL), prolymphocytic leukemia (PLL), large granular lymphocytic (LGL) leukemia, and hairy cell leukemia (HCL). In some embodiments, the cancer is a lymphoma. In some embodiments, the cancer is a non-Hodgkin lymphoma or a Hodgkin's lymphoma. In some embodiments the cancer is selected from the group consisting of follicular lymphoma, Burkitt lymphoma, Waldenström macroglobulinemia, diffuse large B cell lymphoma, primary mediastinal B cell lymphoma, small lymphocytic lymphoma, marginal zone lymphoma, mantle cell lymphoma, peripheral T cell lymphoma (not otherwise specified), anaplastic large cell lymphoma, angioimmunoblastic lymphoma, and cutaneous T cell lymphoma. In some embodiments the cancer is selected from the group consisting of nodular sclerosis Hodgkin lymphoma, mixed cellularity Hodgkin lymphoma, lymphocyte-rich Hodgkin's disease, and lymphocyte-depleted Hodgkin's disease. In some embodiments, the method predicts, diagnoses, or monitors a dermatological disease or disorder in a subject in need thereof. In some embodiments, the dermatological disease or disorder is dermatological disease or disorder is psoriasis, acne vulgaris, hidradenitis suppurativa, androgenic alopecia, acanthosis nigricans, or atopic dermatitis. In some embodiments, the method predicts, diagnoses, or monitors the risk of cardiovascular disease. In some embodiments, the method predicts, diagnoses, or monitors the risk of diabetes. In some embodiments, the method predicts, diagnoses, or monitors the risk of pre-diabetes.

Methods of this disclosure that determine (e.g., measure, detect) the degree of cellular energy metabolism can further predict, diagnose, or monitor a pathological condition in a subject in need thereof. The disclosure relates to methods of predicting, diagnosing, or monitoring any pathological condition in a subject in need thereof. The pathological condition can be any pathological condition including, but not limited to, acral dry gangrene, carotenosis, diabetic dermopathy, diabetic bulla, diabetic cheiroarthropathy, malum perforans, necrobiosis lipoidica, scleredema, waxy skin, diabetic foot, diabetic foot ulcer, or neuropathic arthropathy. In some embodiments, the method predicts, diagnoses, or monitors a pathological condition in a subject in need thereof. In some embodiments, the pathological condition is acral dry gangrene, carotenosis, diabetic dermopathy, diabetic bulla, diabetic cheiroarthropathy, malum perforans, necrobiosis lipoidica, scleredema, waxy skin, diabetic foot, diabetic foot ulcer, or neuropathic arthropathy.

### DEFINITIONS

All publications and patents cited in this disclosure are incorporated by reference in their entirety. To the extent, the material incorporated by reference contradicts or is inconsistent with this specification, the specification will supersede any such material. The citation of any references herein is not an admission that such references are prior art to the present disclosure. Various terms relating to aspects of the description are used throughout the specification and claims. Such terms are to be given their ordinary meaning in the art unless otherwise indicated. Other specifically defined terms are to be construed in a manner consistent with the definitions provided herein.

The articles “a” and “an” are used herein to refer to one or more than one (e.g., to at least one) of the grammatical object of the article. By way of example, “an element” means one element or more than one element.

The term “about” when referring to a measurable value such as an amount, a temporal duration, and the like, is meant to encompass variations of ±20% or less, or in some instances ±15% or less, or in some instances ±10% or less, or in some instances ±5% or less, or in some instances ±1% or less, or in some instances ±0.1% or less, from the specified value, as such variations are appropriate.

The phrase “and/or” as used herein should be understood to mean “either or both” of the elements so conjoined, e.g., elements that are conjunctively present in some cases and disjunctively present in other cases. Multiple elements listed with “and/or” should be construed in the same fashion, e.g., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The term “effective amount” as used herein refers to an amount of a compound or a pharmaceutical formulation of the compound described herein, which is sufficient to achieve the desired results under the conditions of administration. For example, an effective amount of a compound or a pharmaceutical formulation of the compound described herein for the treatment of an epilepsy disorder is an amount that can manage seizure activity, suppress seizure, allow the patient to recover from a hyperexcitable state, prevents seizure-relapse, or can provide continued suppression of seizure. A skilled clinician can determine appropriate dosing based on a variety of considerations including the severity of the disorder, the subject's age, weight, general health, and other considerations.

As used herein, the terms “treat,” “treatment,” or “treating” and grammatically related terms, refer to an improvement of any sign, symptoms, or consequence of the disease, such as prolonged survival, less morbidity, and/or a lessening of side effects. As is readily appreciated in the art, full eradication of disease is preferred but not a requirement for treatment.

The term “transmitting plane” as used herein refers to a component of a monitoring device for detecting cellular energy metabolism that is able to generate and transmit a radio frequency and corresponding electric field to detect the resistance (e.g., bioimpedance) in a circuit due to a solution comprising cells or a bodily surface within the electric field. Unexpectedly, it was discovered that the bioimpedance at certain radio frequencies is correlated to the degree of cellular energy metabolism (e.g., consumption of glucose, production of adenosine triphosphate).

The term “ground plane” as used herein refers to a component of a monitoring device for detecting cellular energy metabolism. The geometry (e.g., shape) of the ground plane and the gap distance between the transmitting plane(s) and ground plane in a device impact (e.g., adjust, alter, control) the penetration depth of the electric field of radio frequencies to a cellular target (e.g., a solution containing cells, a surface containing cells, a dermal surface of a subject, a mucosal surface of a subject) and the sensitivity of detection of ATP changes in the cellular target.

The term “operating value” as used herein refers to any electrical parameter of a circuit (e.g., a circuit of a monitoring device for detecting cellular energy metabolism) including, but not limited to, voltage (V), current (I), impedance (Z), capacitance (C), bioimpedance, electric power (P), inductance (L), frequency (f), and combinations thereof. Measurement of operating values of the circuit of a device in contact with a solution comprising cells or a bodily surface can be used to calculate the impedance (e.g., bioimpedance) of the solution comprising cells or the bodily surface. This bioimpedance can further be correlated to the degree of cellular energy metabolism (e.g., glucose consumption, ATP production).

The term “biopsy explant” as used herein refers to a cell culture of created from any variety of biopsy (e.g.,) from a subject.

The term “dermal surface” as used herein refers to any surface of a subject covered in skin. Dermal surfaces include, but are not limited to, a hand (e.g., palm, finger), abdomen, arm (e.g., wrist, forearm, shoulder, elbow), chest, back, neck, buttocks, leg (e.g., thigh, calf, shin, ankle), foot (e.g., footpad, toe), head (e.g., ear, face, scalp, nose), and combinations thereof.

The term “mucosal surface” as used herein refers to any surface of a subject covered by epithelium that secretes or is covered with mucus. Mucosal surfaces include, but are not limited, to the aural mucosa (e.g., middle ear), oral mucosa (e.g., frenulum), esophageal mucosa, rectal mucosa, gastric mucosa, intestinal mucosa, respiratory mucosa, vaginal mucosa, urethral mucosa, endometrial mucosa, nasal mucosa (e.g., olfactory mucosa), penile mucosa, conjunctiva mucosa, and combinations thereof.

### EXAMPLES

### Example 1. Determination of the Degree of Cellular Energy Metabolism In Vitro

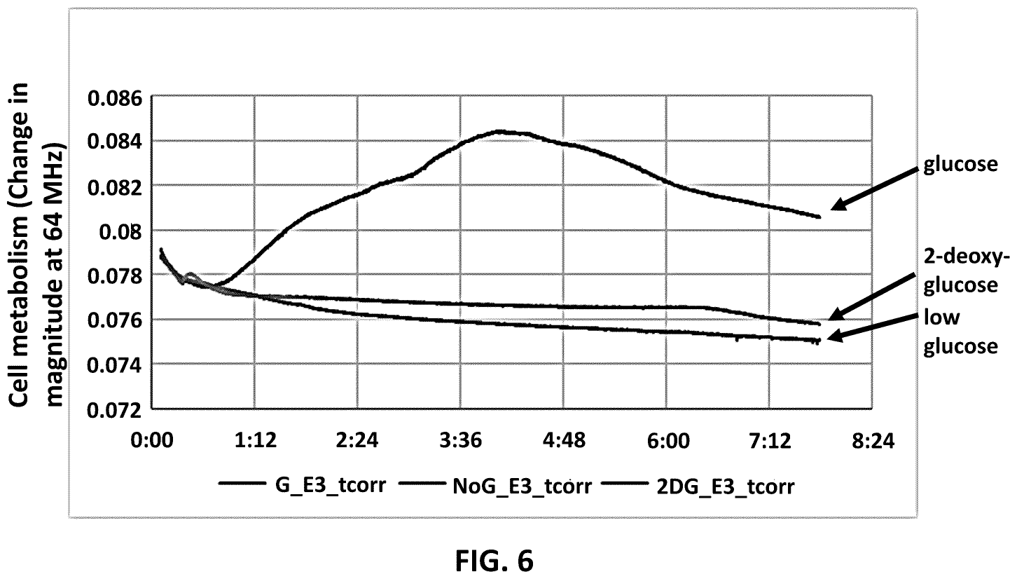

Jurkat T cells were obtained from American Type Culture Collection (ATCC) (#TIB-152) and maintained in high D-Glucose Dulbecco's Modified Eagle Medium (DMEM) (THERMO FISHER SCIENTIFIC INC., #10565042, 17.5 mM/315 mg/dl D-glucose) supplemented with 10% fetal bovine serum (FBS, THERMO FISHER SCIENTIFIC INC., #16140071), penicillin/streptomycin (Pen/Strep), beta-Mercaptoethanol (b-ME) in a 37° C. humidified incubator, 5% CO2. At time point 0:00 (hr.: min.), Jurkat T cells (3×10⁹) were suspended in three separate solutions of low D-glucose DMEM (THERMO FISHER SCIENTIFIC INC., #10565042, 5.5 mM (100 mg/dl D-glucose) supplemented with fetal bovine serum (10% v/v, THERMO FISHER SCIENTIFIC INC. #16140071), penicillin/streptomycin (Pen/Strep), beta-mercaptoethanol (20 ml of media, 5×10⁷ cells per mL each) in tissue culture dishes (10 cm). The cell suspension was placed in an incubator at 37° C. at maximum humidity, carbon dioxide concentration of 5%. Cells were mixed continuously on an orbital shaker (60 rpm). Following 60 minutes of incubation for equilibration, a sensor device of this disclosure was placed in each solution. To the resulting solutions were added 200 μl of media containing 100× D-glucose (e.g., to a final concentration of 200 mg/dl), control media (identical media, but containing low D-glucose (5.5 mM, 100 mg/dl)), and media containing 100× 2-deoxy-D-glucose (identical media, but replacing D-glucose with 2-deoxy-D-glucose at a final concentration of e.g., 200 mg/dl). The radio frequency was detected at 64MHz with the sensor device over the course of approximately 8 h. The radio frequency results are displayed in FIG. 6. While the control media alone showed no detectable radio frequency peak in magnitude at 64 MHz, a broad peak was observed when glucose was supplied to the Jurkat T cells, reaching an apex at approximately 4 h. In contrast, 2-deoxy-D-glucose showed no detectable peak. These data indicate that the sensor device detects ion shifts primarily due to glucose processing and, to a much lesser degree, glucose transport into cells and/or the osmotic effect from glucose addition to the culture medium. Further, the signal is measuring the degree of glucose metabolism inside of cells (e.g., ATP production) as 2-deoxy-D-glucose showed no comparable signal peak to D-glucose.

### Example 2. Serial Feeding Measurement of Interstitial Glucose Verses Cellular Energy Metabolism Determination In Vivo

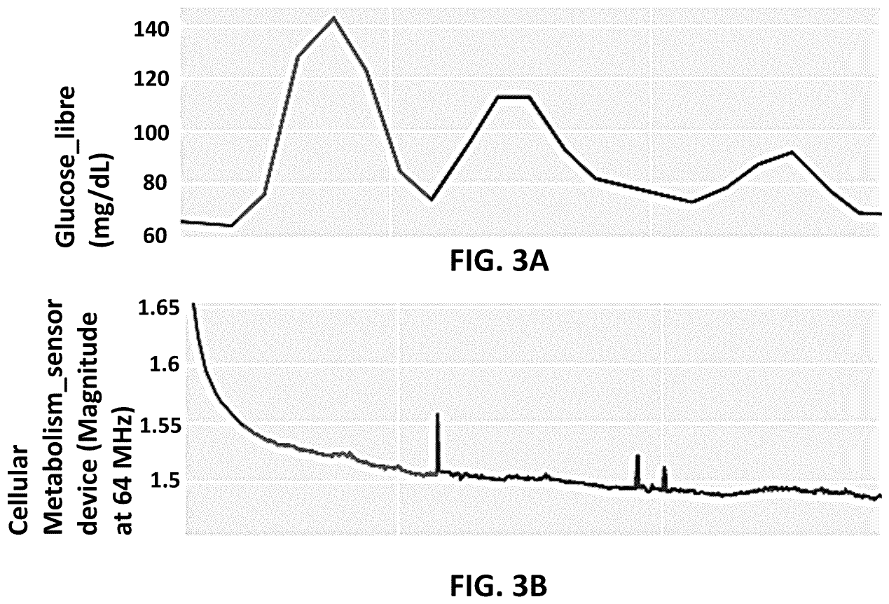

A monitoring device for measuring the degree of cellular energy metabolism was taped onto a subject. To the same subject was applied a Libre® (interstitial glucose sensor). Three blood glucose excursions were detected by the interstitial glucose sensor (see FIG. 3A). In contrast, cellular energy metabolism monitoring device detected cellular energy metabolism concurrent with only the third glucose excursion (see FIG. 3B).

### Example 3. Head-to-head Comparison of Interstitial Glucose Versus Cellular Energy Metabolism Determination

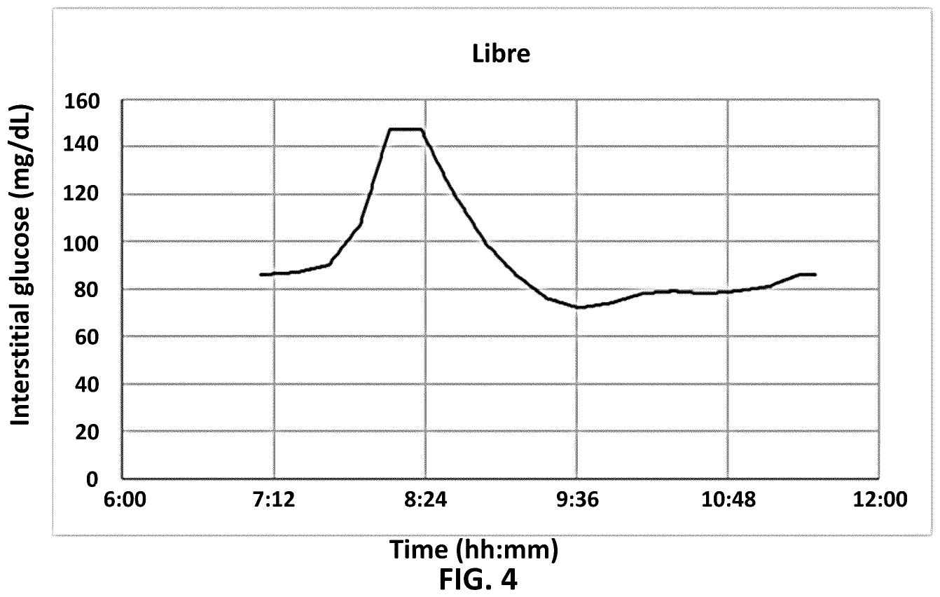

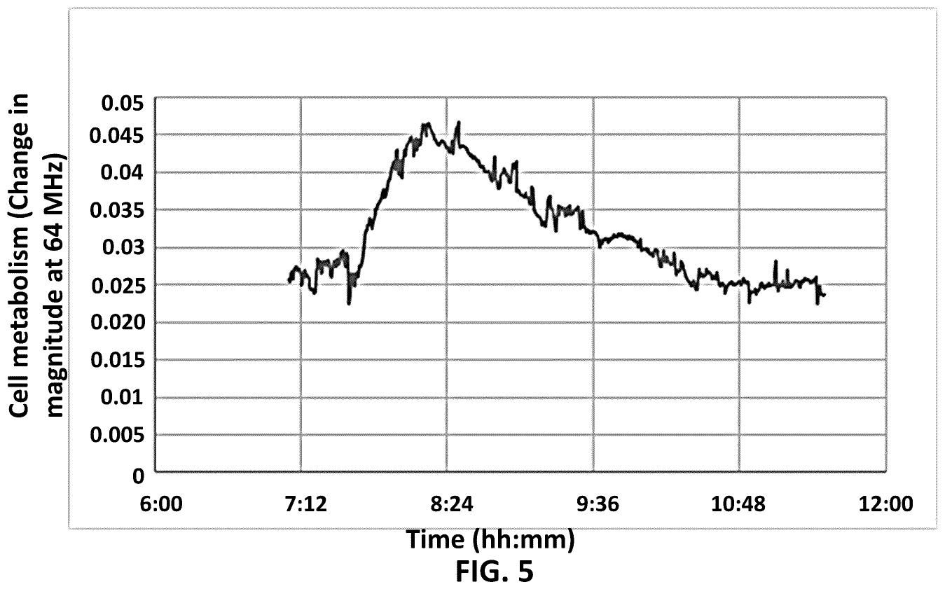

A monitoring device for measuring the degree of cellular energy metabolism was taped onto a subject. To the same subject was applied a Libre® (interstitial glucose sensor). A glucose excursion was detected by the interstitial glucose sensor (see FIG. 4). The monitoring device measured a corresponding increase in cellular energy metabolism (see FIG. 5). The decline in cellular energy metabolism was more gradual than that measured for interstitial glucose concentration. It is believed that this gradual decline is due to cells continuing to consume glucose (and produce intracellular ATP) after the interstitial glucose excursion is complete.

### Example 4. Measurement of Intracellular ATP Concentrations by Luciferase-Based Assay

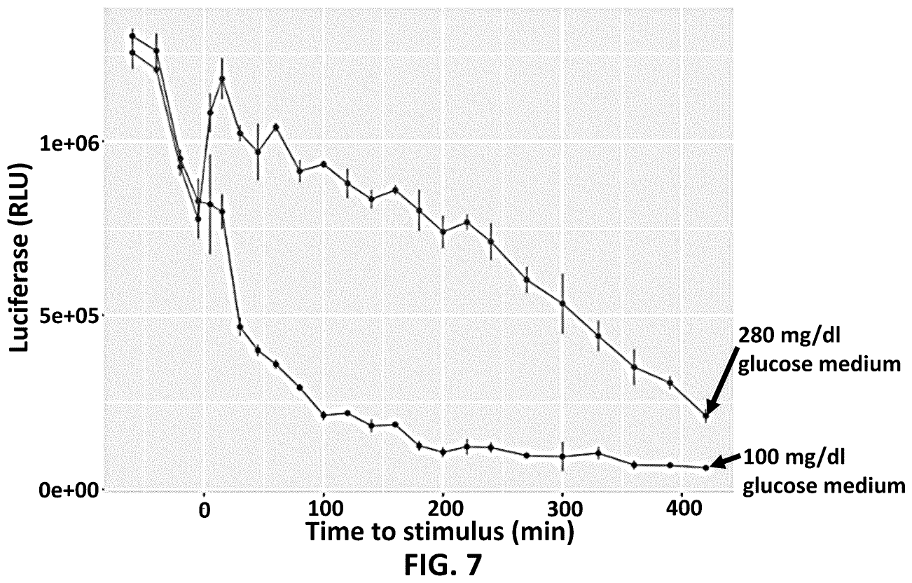

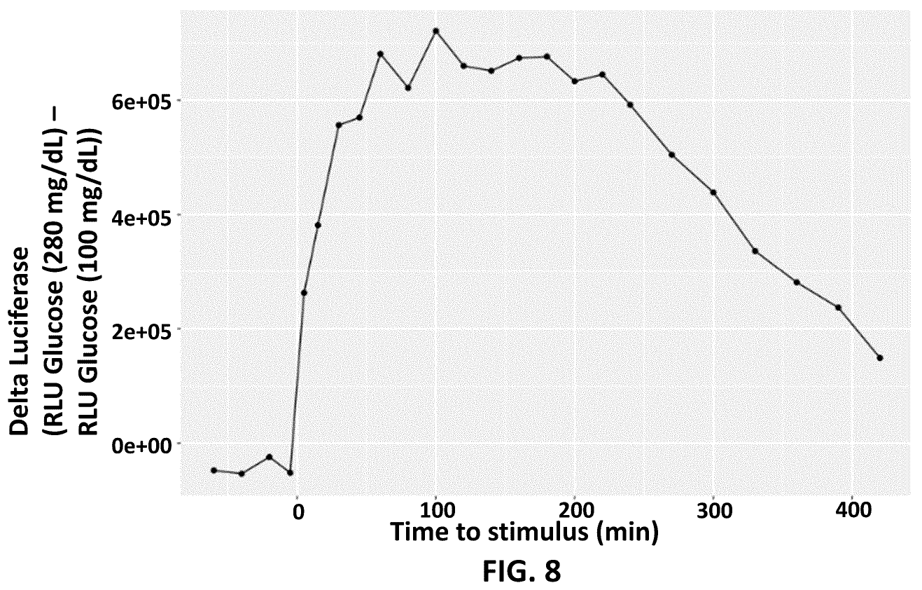

Intracellular ATP concentrations were assessed using a luciferase-based assay to determine the rate of ATP production following addition of glucose. Jurkat T cells (1×10⁷ cells per well in low D-glucose DMEM (100 μl, 5.5 mM, 100 mg/dl D-glucose)) were aliquoted into flat-bottom 96-well plates. Cells were stimulated with DMEM either containing D-glucose (15.5 mM, 280 mg/dl final solution) or low D-glucose (5.5 mM, 100 mg/dl), an effective difference of 10 mM (180 mg/dl) glucose, at time point 0 min. Cells were quantitatively removed from the incubation plate. An aliquot of cell suspension (50 μl) was transferred onto frozen CELLTITER-GLO® 2.0 (PROMEGA CO.) assay reagent on dry ice. The resulting mixture was thawed, and the ATP concentration measured via light emission in a 96-well plate-compatible luminometer. The resulting curve of luciferase activity in relative luminescence units (RLUs) over time (min. following stimulation) is shown in FIG. 7. A plot of the difference in luminescence at each time point shows an increase in net ATP concentration followed by a slower decrease in net ATP concentration over the course of the experiment (see FIG. 8). Glucose transport is rapid as indicated by the rapid increase of ATP concentration following addition of D-glucose.

### Example 5. Measurement of Intracellular ATP Concentrations by Radio Frequency Monitoring Device

The degree of cellular energy metabolism was monitored by radio frequency in Jurkat T cell suspensions to compare to direct intracellular ATP measurement of Example 4. Jurkat T cell suspensions were maintained as in Example 4. A radio frequency monitoring device was placed in the cell culture and monitored at 64 MHz frequency as a function of time. Cells were stimulated by addition of (a) D-glucose (180 mg/dl final concentration in DMEM), (b) 2-deoxy-D-glucose (2DG, 180 mg/dl final concentration in DMEM), (c) D-glucose (180 mg/dl final concentration) and BAY-876 (a glucose transporter 1 inhibitor, 5 μM final concentration), and (d) DMEM with low D-glucose (5.5 mM, 100 mg/dl, ‘plain medium’). All stimulations were conducted with the same volume of liquid.

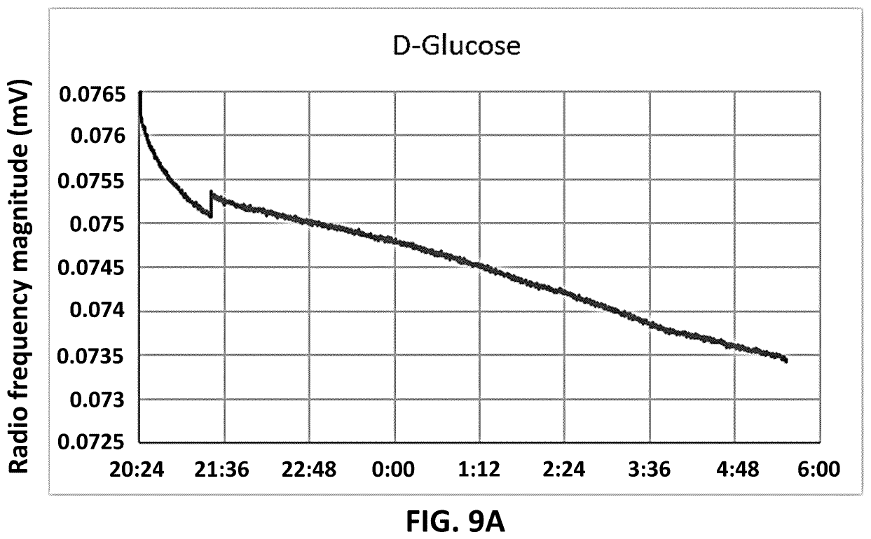

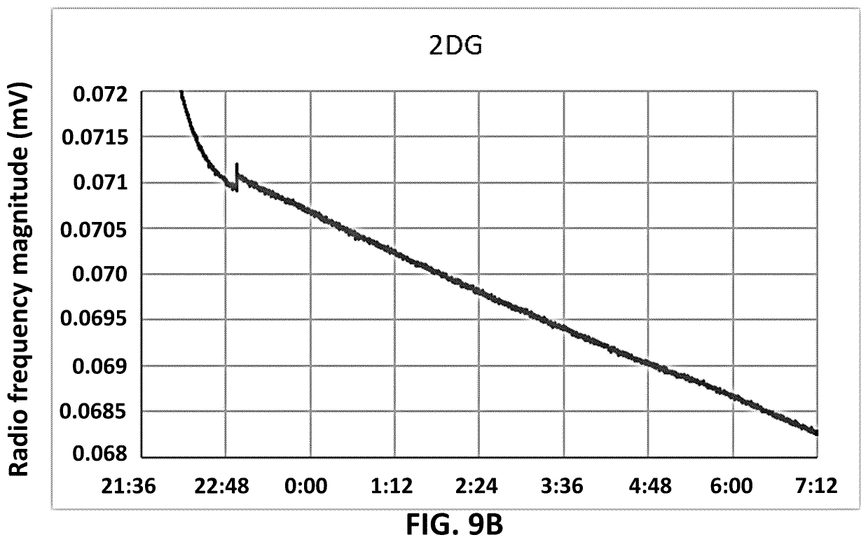

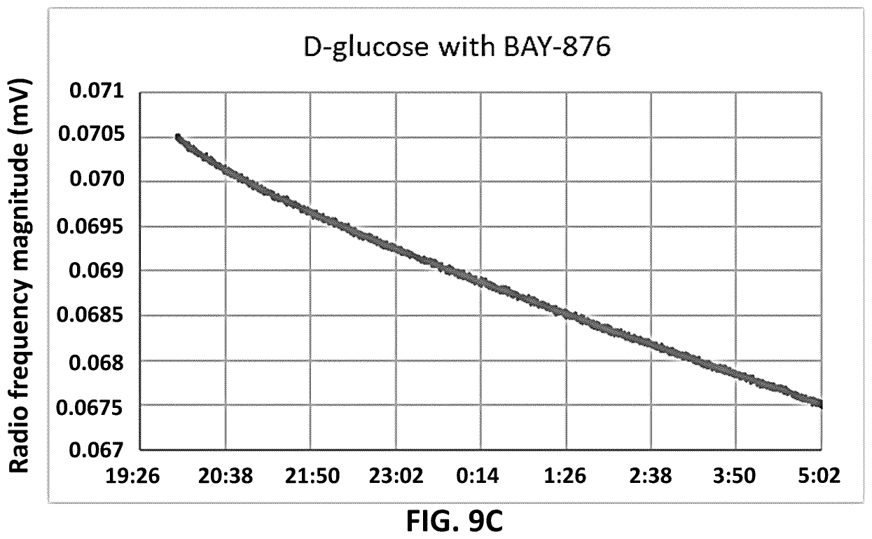

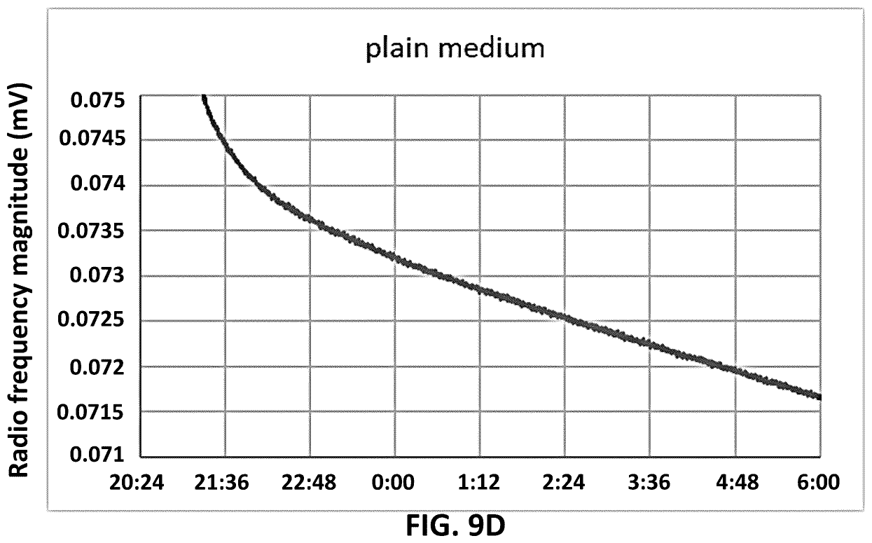

Plain medium (DMEM with low D-glucose (5.5 mM, 100 mg/dl)) showed a change in magnitude at 64 MHz that is initially rapid, due to a change in incubator interior temperature by an increase of 2°° C., followed by a slowing, linear decay. The linear decay is due to the depletion of ATP in cells (see FIG. 9D). The addition of D-glucose in the presence of BAY-876 also shows a smooth decline becoming linear over time (see FIG. 9C). The less rapid initial decay in the experiment is due to the incubator being close to its target temperature when D-glucose is added (interior incubator temperature increases 0.3° C.). BAY-876 inhibits the uptake of D-glucose into cells, causing an osmotic effect. However, this effect does not substantially impact the curve shape from that of ‘plain medium’ addition shown in FIG. 9D, indicating that observed changes in radio frequency magnitude are not primarily due to osmotic changes or changes in extracellular glucose. In contrast, the magnitude at 64 MHz following stimulation with D-glucose (FIG. 9A) shows both a peak shortly after addition of stimulus, and a more gradual decline than that of ‘plain medium’ (low D-glucose) (FIG. 9C) or D-glucose with BAY-876 (FIG. 9D). While 2DG stimulus leads to an initial peak, similar to D-glucose, the magnitude at 64 MHz quickly adopts a similar linear decline as seen in ‘plain medium’ and with BAY-876 (see FIG. 9B).

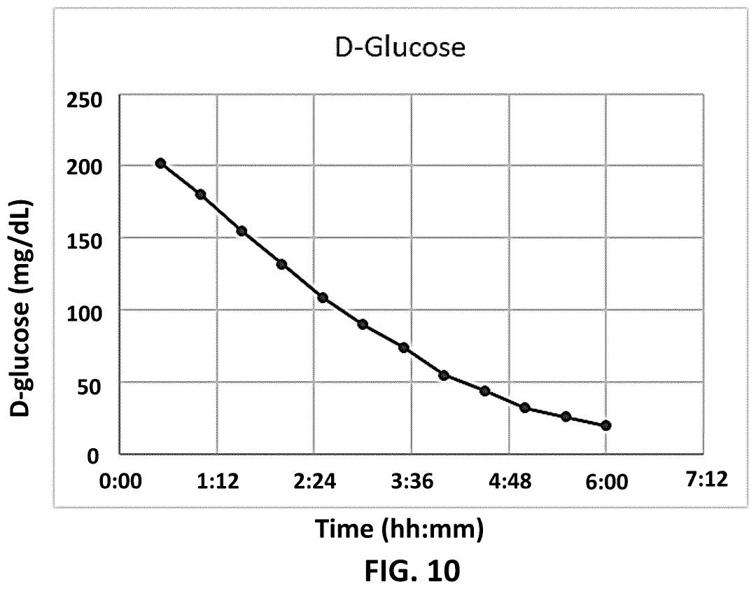

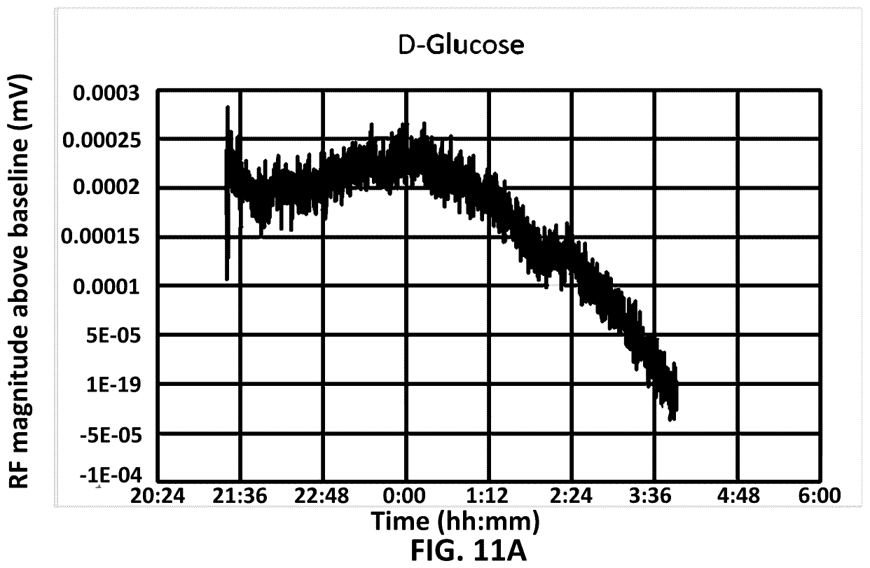

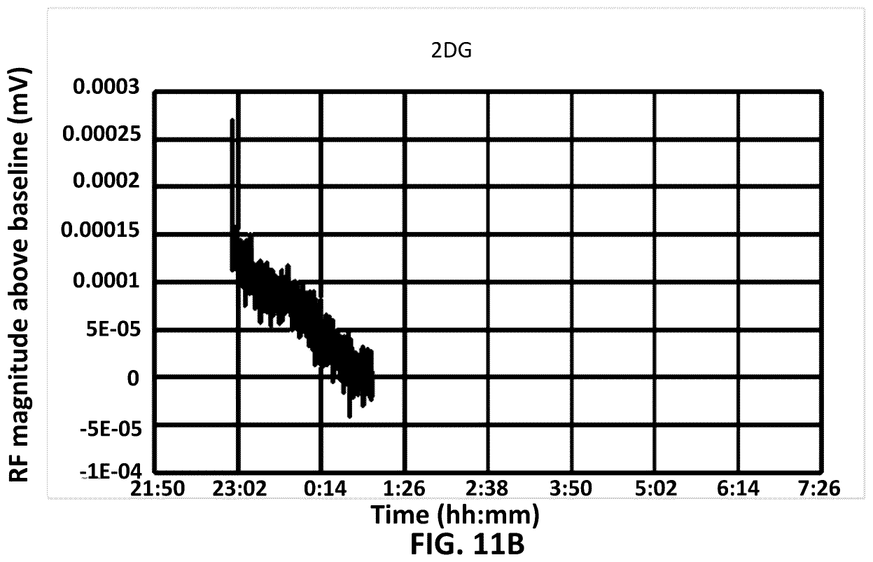

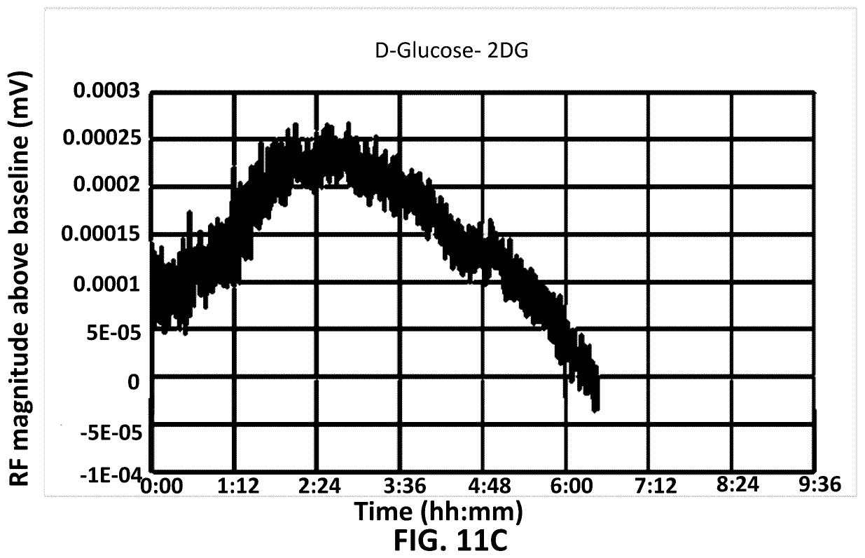

To determine the net effects of both the D-glucose (FIG. 9A) and 2DG (FIG. 9B), the concentration D-glucose was determined. D-glucose is rapidly depleted over the course of the experiment as measured by media D-glucose concentrations (FIG. 10), reaching almost 0 mg/dl by 6 hr. following stimulation, indicating a similar baseline between the two stimuli and ‘plain medium’ with low D-glucose or 2DG. The net change in radio frequency magnitude by D-glucose stimulation and 2DG stimulation were determined by subtracting DMEM with low D-glucose (5.5 mM, 100 mg/dl D-glucose, ‘plain medium’) as the background. D-glucose stimulation showed an initial net increase in magnitude followed by a slow net decrease (see FIG. 11A). In contrast, 2DG stimulation showed only an initial net increase between about 23:02 and 1:00 (see FIG. 11B). As 2DG and D-glucose are both transported into cells, this suggests that transportation of D-glucose or 2DG results in a short-term initial increase in magnitude of frequency at 64 MHz. However, as glucose concentrations change slowly in vivo (e.g., from zero to peaking over the course of an hour) this transport effect is not anticipated to be observed in live subjects. The difference between the net effects of D-glucose and 2DG stimuli shows a curve that rises and falls over the course of 6 hours (see FIG. 11C). This curve closely resembles the shape of net ATP measured by luciferase assay (FIG. 8), indicating that the dominant contribution of change in net magnitude in radio frequency is due to the change in intracellular ATP concentration (e.g., cellular energy metabolism) or a cellular process that tracks it.

1. A method of non-invasively measuring the degree of cellular energy metabolism on a bodily surface or solution comprising cells, the method comprising:
a) contacting a monitoring device for detecting the degree of cellular energy metabolism to a bodily surface or a solution comprising cells, wherein the monitoring device for detecting the degree of cellular energy metabolism comprises a sensor substrate that comprises
i) one or more transmitting planes for transmitting a transmitted signal at a radio frequency, and
ii) a ground plane with a gap between the ground plane and the one or more transmitting planes for controlling the penetration depth of an electric field of the transmitted signal and adjusting sensitivity to adenosine triphosphate changes on the bodily surface or the solution comprising cells,
b) generating the transmitted signal at the radio frequency,
c) measuring one or more operating values of the circuitry of the monitoring device at the radio frequency of the transmitted signal in contact with the bodily surface or the solution comprising cells,
d) maintaining contact with the bodily surface or the solution comprising cells for a desired length of time, and
e) collecting the impedance data, the voltage data, or the current data from the sensor substrate of the monitoring device that indicates a degree of cellular energy metabolism for the bodily surface or the solution comprising cells.

2. The method of claim 1, wherein the radio frequency of the transmitted signal ranges from about 0.5 MHz to about 250 MHz, or about 40 MHz to about 75 MHz.

3. (canceled)

4. The method of claim 1, wherein the radio frequency of the transmitted signal is about 64 MHz.

5. The method of claim 1, wherein the monitoring device comprises one transmitting plane or two transmitting planes.

6. (canceled)

7. The method of claim 1, wherein one or more transmitting planes is bar shaped or annular.

8. The method of claim 7, wherein the bar shaped transmitting plane has a width ranging from about 0.25 mm to about 15 mm, or about 1 mm to about 8 mm.

9. (canceled)

10. (canceled)

11. The method of claim 1, wherein the desired length of time ranges from about 1 minute to about 24 hours, or about 1 minute to about 2 hours.

12. (canceled)

13. (canceled)

14. The method of claim 1, wherein the sensor substrate is separated from the bodily surface or solution comprising cells by an insulative layer.

15. The method of claim 14, wherein the thickness of the insulative layer is about 10 μm or less.

16. The method of claim 1, wherein the method predicts, diagnoses, or monitors a cardiovascular disease in a subject in need thereof.

17. The method of claim 16, wherein the cardiovascular disease is coronary artery disease, peripheral arterial disease, cerebrovascular disease, renal artery stenosis, or aortic aneurysm.

18. The method of claim 1, wherein the method predicts, diagnoses, or monitors a metabolic disease or disorder in a subject in need thereof.

19. The method of claim 18, wherein the metabolic disease or disorder is prediabetes, type 1 diabetes, type 2 diabetes, glycogen storage disease, galactosemia, or cancer.

20-24. (canceled)

25. The method of claim 1, wherein the method predicts, diagnoses, or monitors a dermatological disease or disorder in a subject in need thereof.

26. The method of claim 25, wherein the dermatological disease or disorder is psoriasis, acne vulgaris, hidradenitis suppurativa, androgenic alopecia, acanthosis nigricans, or atopic dermatitis.

27. The method of claim 1, wherein the method predicts, diagnoses, or monitors a pathological condition in a subject in need thereof.

28. The method of claim 27, wherein the pathological condition is acral dry gangrene, carotenosis, diabetic dermopathy, diabetic bulla, diabetic cheiroarthropathy, malum perforans, necrobiosis lipoidica, scleredema, waxy skin, diabetic foot, diabetic foot ulcer, or neuropathic arthropathy.

29. (canceled)

30. The method of claim 1, wherein the bodily surface comprising cells is an epidermis, a dermis, a subcutaneous tissue, or a combination thereof of a dermal surface of a subject.

31. The method of claim 1, wherein the bodily surface comprising cells is an epithelial layer, a lamina propria, a muscularis mucosa, a submucosal layer, a muscle, or a combination thereof of a mucosal surface of a subject.

32. The method of claim 1, wherein the solution comprising cells is a cell culture.

33. (canceled)
